(19) United States
(12) Patent Application Publication
(10) Pub. No.: US 2025/0221755 A1
(43) Pub. Date: Jul. 10, 2025

(54) Treatment of Living Biological Surfaces with Plasma

(71) Applicant: Era Medical Ltd., Windsor (GB)

(72) Inventors: Alexander BRITVA, Migdal Ha'Emek (IL); Guy GOUDSMIT, Windsor (GB)

(21) Appl. No.: 19/008,382

(22) Filed: Jan. 2, 2025

(30) Foreign Application Priority Data
Jan. 4, 2024 (GB) 2400130.7

(51) Int. Cl. A61B 18/04; A61B 18/00

(52) U.S. Cl. CPC A61B 18/04; A61B 2018/00071; A61B 2018/00583; A61B 2018/00922

(57) ABSTRACT

Disclosed are methods and devices suitable for the treatment of a surface with cold plasma, especially the surface of living biological tissue where the cold plasma is generated in proximity of the surface.

### RELATED APPLICATION

The present application gains priority from UK Patent Application GB 2400130.7 filed Jan. 4, 2024, which is included by reference as if fully set-forth herein.

FIELD AND BACKGROUND OF THE INVENTION

The invention, in some embodiments, relates to the field of surface treatment with cold plasma, especially treatment of the surface of living biological tissue, in some embodiments the non-therapeutic (cosmetic) treatment of the surface of living biological tissue and in other embodiments the therapeutic (medical) treatment of the surface of living biological tissue. In some embodiments, the invention relates to devices suitable for generating cold plasma that is exceptionally suitable for treatment of surfaces, especially for treating the surface of living biological tissue.

Plasma is a highly electrically-conductive state of matter comprising ionized and excited particles. Cold plasma is a plasma which electron temperature is very high while the temperature of the heavy species in the plasma is relatively low.

The use of cold plasma for treatment of surfaces is well known. For example, a gas (helium, argon) is driven to flow into the distal end of a quartz tube (e.g., 2 to 3 mm inner diameter) with an upstream electrode and a downstream electrode. An alternating current (e.g., 15 to 25 kV at 4 to 6 kHz) is applied between the two electrodes to ionize some of the gas in the quartz tube thereby forming a cold plasma inside the tube. The gas-flow carries ionized species entrained in the gas-flow as a plasma plume. It has been found that such a plasma plume can be directed to a skin surface for medical treatment (e.g., of warts) and non-therapeutic treatments (e.g., skin-tightening). A commercially-available device that generates a plasma plume is the Planoplas® by Müller German Beauty Tech UG (Karlsruhe, Germany).

It would be useful to treat additional conditions, both medical treatments and non-therapeutic treatments, with cold plasma. It would be useful to have a device suitable for generating cold plasma to more effectively treat surfaces.

### SUMMARY OF THE INVENTION

Some embodiments of the invention relate to surface treatment with cold plasma, especially treatment of the surface of living biological tissue, in some embodiments the non-therapeutic (cosmetic) treatment of the surface of living biological tissue and in other embodiments the therapeutic (medical) treatment of the surface of living biological tissue. In some embodiments, the invention relates to devices suitable for generating cold plasma that is exceptionally suitable for treatment of surfaces, especially for treating the surface of living biological tissue.

According to an aspect of some embodiments of the teachings herein there is provided a method of treating living biological tissue of a subject, comprising:
providing an electrode having a longitudinal axis and a distal end;
in the presence of a gaseous atmosphere, placing the distal end of the electrode in proximity of a surface of living tissue with the longitudinal axis directed towards the surface so that the distal end of the electrode is at a distance of not less than about 1 mm and not more than about 10 mm from the surface so as to form a gap between the distal end of the electrode and the surface wherein the surface thereby functions as a ground electrode; and
supplying to the electrode a potential alternating at a rate of not less than about 5 MHz with a power of not less than about 1 W and not more than about 100 W,
   wherein the potential supplied to the electrode exceeds a breakdown potential of the gap thereby generating cold plasma in the gap in proximity of the surface which generated cold plasma treats the biological tissue. In some embodiments, the treatment is a non-therapeutic treatment for treating a cosmetic condition. Alternatively, in some embodiments the treatment is a therapeutic treatment, e.g., for treating a medical condition.

In some embodiments, generating cold plasma in proximity of the surface is accompanied by formation of plasma streamers between the distal end of the electrode and the surface.

In some embodiments, the surface is a scalp and the non-therapeutic treatment is for stimulating hair growth from the scalp.

In some embodiments, the gaseous atmosphere is at least about 50 mole percent Ar. In some embodiments, the gaseous atmosphere is between about 0.1 and about 10 mole percent N₂ and between about 0.1 and about 10 mole percent O₂.

In some embodiments, the distance is not more than about 8 mm, and in some preferred embodiments even not more than about 10 mm.

In some embodiments, the cross-sectional size of the distal end of the electrode that is perpendicular to the longitudinal axis is not less than about 0.20 cm² (equivalent to a 0.5 cm diameter circle), not less than about 0.8 cm² (equivalent to a 1 cm diameter circle). In some embodiments, the cross-sectional size of the distal end of the electrode that is perpendicular to the longitudinal axis is not more than about 80 cm² (equivalent to a 10 cm diameter circle).

In some embodiments, the magnitude of the potential is not less than about 1 kV and not more than about 10 kV.

In some embodiments, the rate at which the potential alternates is not less than about 8 MHz, not less than 9 MHz and even not less than 10 MHz.

In some embodiments, the rate at which the potential alternates is not more than about 600 MHz, not more than about 433 MHz and even not more than about 300 MHz. In some embodiments, the power is not less than about 5 W and not more than about 80 W.

According to an aspect of some embodiments of the teachings herein there is also provided a device suitable for treating living biological tissue by generating cold plasma in proximity of the surface of the biological tissue, the device comprising:
a. an electrode having a longitudinal axis, a proximal end and a distal end, the electrode being part of a circuit;
b. an offset component physically associated with the electrode, the offset component having a distal end, configured so that when the distal end of the offset component contacts a surface of living biological tissue, the distal end of the electrode is maintained at a distance of not less than about 1 mm and more than about 10 mm from the surface, thereby forming a gap between the distal end of the electrode and the surface; and
c. a gas inlet configured for functional association with a gas reservoir,
   wherein the device is configured so that when the distal end of the offset component contacts a surface, gas exiting the gas inlet fills the gap between the distal end of the electrode and the surface.

The device is configured to allow generation of cold plasma in the gap in proximity of the surface of living biological tissue where the surface functions as a ground electrode and when a potential supplied to the electrode exceeds a breakdown potential of the gap. Without wishing to be held to any one theory, it is believed that the cold plasma is generated by a glow discharge mechanism.

In some embodiments, the circuit is an open circuit.

In some embodiments, the electrode, the offset component and the gas inlet are all components of a handpiece of the device.

In some embodiments, the distance is not than about 8 mm and in some embodiments the distance is not more than about 5 mm.

In some embodiments, the surface is devoid of any discontinuity (e.g., is flat and smooth). In some preferred embodiments, the distal end of the electrode comprises at least one discontinuity. In some such embodiments, the distal end of the electrode comprises not less than four discontinuities.

In some embodiments, the cross-sectional size of the distal end of the electrode that is perpendicular to the longitudinal axis is not less than about 0.20 cm² and in some embodiments not less than about 0.8 cm². In some embodiments, the cross-sectional size of the distal end of the electrode that is perpendicular to the longitudinal axis is not more than about 80 cm².

In some embodiments, the distal end of the offset component is configured to allow benign sliding along the surface of living biological tissue.

In some embodiments, the offset component comprises an enclosure having a wall defining a closed proximal portion and an open distal portion comprising a rim, thereby defining an open-ended hollow, wherein the distal end of the electrode is located inside the open-ended hollow and is recessed relative to the rim so that when the rim contacts a surface, the distal end of the electrode is at the distance from the surface.

In some embodiments, the gas inlet is directed into the open-ended hollow so that gas exiting the gas inlet enters the open-ended hollow.

In some embodiments, the device further comprises an AC power supply circuit functionally associated with the electrode, the AC power supply circuit configured to supply a potential alternating at a rate of not less than about 5 MHz, alternatively not less than about 8 MHZ, not less than about 9 MHz and alternatively even not less than about 10 MHz to the electrode. In some such embodiments, the AC power supply circuit is configured to supply a potential alternating at a rate of not more than about 600 MHz, not more than about 433 MHz and in some embodiments not more than about 300 MHz to the electrode.

In some embodiments, the AC power supply circuit is configured to supply a potential of not less than about 1 kV relative to ground to the electrode.

In some embodiments, the AC power supply circuit is configured to supply a potential of not more than about 10 kV relative to ground to the electrode.

In some embodiments, the AC power supply circuit is configured to supply the potential to the electrode with a power of not less than about 1 W and not more than about 100 W.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 schematically depicts an embodiment of a device according to the teachings herein.

### DESCRIPTION OF SOME EMBODIMENTS OF THE INVENTION

Some embodiments of the invention relate to surface treatment with cold plasma, especially treatment of the surface of living biological tissue. In some embodiments, the treatment is the non-therapeutic treatment of the surface of living biological tissue. In some embodiments, the treatment is therapeutic treatment of the surface of living biological tissue. In some embodiments, the invention relates to devices suitable for generating cold plasma that is exceptionally suitable for treatment of surfaces, especially for treating the surface of living biological tissue.

The principles, uses and implementations of the teachings herein may be better understood with reference to the accompanying description and figures. Upon perusal of the description and figures present herein, one skilled in the art is able to implement the invention without undue effort or experimentation. In the figures, like reference numerals refer to like parts throughout.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

### Methods According to the Teachings Herein

### Generation of Cold Plasma in a Nitrogen/Oxygen Atmosphere

According to an aspect of some embodiments of the teachings herein, there is provided method of treating living biological tissue of a subject, comprising: generating cold plasma in a gaseous atmosphere, the gaseous atmosphere comprising between about 0.1 and about 10 mole percent nitrogen gas and between about 0.1 and about 10 mole percent oxygen gas; and contacting the generated cold plasma with a surface of living biological tissue, thereby treating the living biological tissue. The cold plasma is generated in any suitable way using any suitable device including prior art methods and devices as well as the novel methods and devices disclosed herein. Without wishing to be held to any one theory, it is currently believed that generating a cold plasma in such an atmosphere produces NO (nitric oxide), a molecule known for having various biological functions including as a vasodilator. For some reason, the produced NO efficiently penetrates the surface of the living biological tissue to provide a biological effect. In some embodiments, at least about 50 mole percent, at least about 60 mole percent and even more preferably at least about 80 mole percent of the gaseous atmosphere is a noble gas, especially argon.

In some embodiments, the method is a therapeutic treatment for treating a medical condition. In some such embodiments, the subject is a human. Alternatively, in some embodiments the subject is a non-human animal. In some embodiments, the medical condition is a medical condition which is susceptible to increased blood flow underneath the surface. In some embodiments, the surface is skin. In some embodiments, the medical condition is reduced blood flow in an extremity, for example, in a subject suffering from diabetes.

In some embodiments, the method is a non-therapeutic treatment for treating a cosmetic condition. In some embodiments, the cosmetic condition is a cosmetic condition which is susceptible to increased blood flow underneath the surface. In some such embodiments, the surface is skin (e.g., of the face, neck, décolleté, breast, belly, thighs, buttocks) and the non-therapeutic treatment is for improving skin health and/or skin rejuvenation, for example, treating wrinkles, thin skin and/or saggy skin. In some such embodiments, the surface is skin, preferably of the scalp. In preferred such embodiments, the non-therapeutic treatment is for stimulating hair growth, for example, for treating hair loss (e.g., male-pattern hair loss and female-pattern hair loss), baldness, sparse hair and/or thin hair.

In some embodiments, the surface is skin, preferably of the scalp. In some such embodiments, the condition is at least one of hair loss, baldness, thin hair, thin skin, wrinkles and saggy skin.

Cold plasma for Treating Hair Loss and Related Conditions.

According to an aspect of some embodiments of the teachings herein, there is also provided a non-therapeutic method of treating living biological tissue of a subject, comprising: generating cold plasma in a gaseous atmosphere; and contacting the generated cold plasma with a skin surface of a subject, thereby stimulating hair growth, e.g., for treating hair loss, baldness, sparse hair or thin hair. The cold plasma is generated in any suitable way using any suitable device including prior art methods and devices as well as the novel methods and devices disclosed herein. In some embodiments, the contact with cold plasma causes at least one of increased hair density (hairs/cm²) growing from the skin surface and increasing the average diameter of hair strands growing from the skin surface. In some such embodiments, the subject is a human. In some alternative such embodiments, the subject is a non-human animal.

### Generation of Cold Plasma in the Proximity of a Surface

According to an aspect of some embodiments of the teachings herein, there is also provided a method of treating living biological tissue of a subject, comprising:
providing an electrode having a longitudinal axis and a distal end;
in the presence of a gaseous atmosphere, placing the distal end of the electrode in proximity of a surface of living tissue with the longitudinal axis directed towards the surface so that the distal end of the electrode is at a distance of not less than about 1 mm and not more than about 10 mm from the surface so as to form a gap between the distal end of the electrode and the surface wherein the surface thereby functions as a ground electrode; and
supplying to the electrode a potential alternating at a rate of not less than about 5 MHz with a power of not less than about 1 W and not more than about 100 W,
   wherein the potential supplied to the electrode exceeds the breakdown potential of the gap thereby generating cold plasma in the gap in proximity of the surface, in preferred embodiments generating the cold plasma at the surface itself. The generated cold plasma treats the biological tissue. In preferred embodiments, generating the cold plasma in proximity of the surface is accompanied by formation of plasma streamers between the distal end of the electrode and the surface.

Unlike known methods of treating living biological tissue with cold plasma, the method of the teachings herein does not require transporting generated cold plasma to the surface of the tissue, for example, entrained in a gas as a plasma stream as discussed in the introduction. Instead, the cold plasma is generated in the gap itself, in some embodiments within about 5 mm of the surface, within about 4 mm of the surface, within about 3 mm of the surface, within about 2 mm of the surface, within about 1 mm surface and even at the surface itself.

The gaseous atmosphere in the gap is any suitable gaseous atmosphere. In some embodiments, the gaseous atmosphere comprises at least about 50 mole percent, at least about 60 mole percent, at least about 80 mole percent, at least about 90 mole percent and even at least about 95 mole percent of a gas selected from the group consisting of He, Ne, O₂, N₂, Ar and Xe, most preferably Ar.

Additionally or alternatively, in some preferred embodiments the gaseous atmosphere comprises between about 0.1 and about 10 mole percent N₂ and between about 0.1 and about 10 mole percent O₂. In a particularly preferred such embodiment, the gaseous atmosphere comprises at least about 50 mole percent, at least about 60 mole percent and even more preferably at least about 80 mole percent noble gas (most preferably Ar, between about 0.1 and about 10 mole percent N₂ and between about 0.1 and about 10 mole percent O₂.

The pressure of the gaseous atmosphere is any suitable pressure. In some embodiments, the pressure is within 10% of ambient (atmospheric) pressure which is dependent on weather conditions and the altitude. In some alternative embodiments, the pressure is lower than atmospheric pressure, in some such embodiments not less than about 10% of ambient (atmospheric) pressure.

In some embodiments, the method is a therapeutic treatment for treating a medical condition. In some such embodiments, the subject is human. Alternatively, in some embodiments the subject is a non-human animal.

In some embodiments, the surface is skin.

In some embodiments, the medical condition is a medical condition which is susceptible to increased blood flow underneath the surface. In some embodiments, the medical condition is reduced blood flow in an extremity, for example, in a subject suffering from diabetes.

In some embodiments, the surface is skin, and the medical condition is selected from the group consisting of acne, atopic dermatitis, epidermolysis bullosa, hidradenitis suppurativa, psoriasis, Raynaud's Phenomenon and scleroderma.

In some embodiments, the surface is skin, and the medical condition is cancer-related, for example, a cancer (e.g., melanoma, basal cell carcinoma, squamous cell carcinoma), a pre-cancerous tumor, a pre-cancerous lesion (e.g., actinic keratoses).

In some embodiments, the medical condition is a wound on the surface, especially a surface is skin, for example, a surgical wound or an ulcer (for example, related to diabetes).

In some embodiments, the medical condition is related to an infection by an organism and the treatment is for at least one of reduction of the organism load and mitigating effects of the infection. In some embodiments, the infection is a bacterial infection. In some embodiments, the medical condition is a lesion related to a viral infection (e.g., warts). In some embodiments, the medical condition is related to a fungal infection (e.g., onychomycosis). In some embodiments, the medical condition is related to a lesion related to a protozoan infection (e.g., leishmaniasis). In some embodiments, the surface is skin and the medical condition is an arthropod infestation, for example, by mites (scabies), lice or fleas.

In some embodiments, the method is a non-therapeutic treatment for treating a cosmetic condition. In some such embodiments, the subject is human. Alternatively, in some embodiments the subject is a non-human animal.

In some embodiments of the non-therapeutic treatment, the surface is skin (e.g., skin of the face, neck, décolleté, breast, belly, thighs or buttocks) and the non-therapeutic treatment is for improving skin health and/or skin rejuvenation, for example, treating wrinkles, thin skin and/or saggy skin. In some such embodiments, the treatment works by one or more mechanisms of fibroblast proliferation, collagen fiber synthesis and increasing epidermal thickness.

In some embodiments of the non-therapeutic treatment, the surface is skin, and the non-therapeutic treatment is for mitigating the appearance of a rosacea outbreak.

In some embodiments, the surface is skin, preferably of the scalp and the non-therapeutic treatment is for stimulating hair growth, for example, for treating hair loss, baldness, sparse hair and/or thin hair. In some embodiments, the treatment causes at least one of increased hair density (hairs/cm²) growing from the treated skin surface (particularly suitable for treating both MPHL (male-pattern hair loss) and FPHL (female-pattern hair loss); and increasing the average diameter of hair strands growing from the treated skin surface. The inventor hypothesized that treatment of a scalp with cold plasma might reduce the rate of or even stop hair loss in men exhibiting early stages of MPHL. As detailed in the Experimental Section, unexpectedly, the non-therapeutic treatment of a scalp with cold plasma according to the teachings herein of 10 men exhibiting MPHL showed a marked increase of hair density rather than simply reducing the rate of hair loss. Even more unexpectedly, an increase in the average diameter of hair strands growing from the treated skin surface is observed.

As discussed in the Background Section, it is known to generate cold plasma in a reactor (e.g., in a quartz tube) and then to transport ionized species to a surface entrained in a gas flow as a plasma plume. According to the teachings herein, cold plasma is not generated and then transported to the surface, but is instead generated in the gap between the distal end of the electrode and the surface to be treated, at the surface or in proximity thereof. Without wishing to be held to any one theory, it is currently believed that embodiments of the teachings herein induce one or more biological effects to the surface or in the tissue underlying the surface, for example by providing:
a comparatively higher concentration of active species at the surface because the cold plasma is generated at or near the surface rather than being transported thereto;
active species having a lifetime that is so short that it precludes transport to the skin surface are generated near and therefore present at the surface;
an electric current that passes from the electrode to the surface; and
UV light that is emitted during the cold plasma generation.

Without wishing to be held to any one theory, it is currently believed that in some embodiments the induced biological effects include at least one of:
increased blood flow.
improvement of sub-surface microcirculation, e.g., in hair follicles, which in turn induces arteriolar vasodilation and angiogenesis.
conversion of existing follicles into the anagen stage; and
reversal of follicle miniaturization.

Further, in preferred embodiments the generating of cold plasma is accompanied by formation of plasma streamers between the electrode and the surface. Compared to cold plasma, streamers are characterized as having a substantially higher current density, greater ionization, higher electron temperature, higher gas temperature and higher excitation rate. As a result, it is currently believed that the streamer-surface points of contact are particularly rich in active species, some such species not being present in the cold plasma. As noted above, in preferred embodiments the gaseous atmosphere comprises at least about 50 mole percent Ar. It has been experimentally observed that, unlike in some other gases, in a gaseous atmosphere that comprises at least about 50 mole percent Ar, the generated streamers continuously move over a surface. Without wishing to be held to any one theory, at least one advantage of a gaseous atmosphere that comprises at least about 50 mole percent Ar is that by continuously moving the generated streamers substantially homogeneously contact all portions of a treated surface.

Further, in preferred embodiments where the gaseous atmosphere is between about 0.1 and about 10 mole percent N₂ and between about 0.1 and about 10 mole percent O₂, it is believed that concurrently with the generation of cold plasma, gaseous NO (nitric oxide) is formed and for some reason efficiently penetrates the surface. NO is known to have many biological functions that may induce useful biological effects such as vasodilation and increased blood flow.

### Distance of Gap

As noted above, the distance between the distal end of the electrode and the surface is not less than about 1 mm and not more than about 10 mm. In some embodiments the distance is not more than about 8 mm and in some embodiments even not more than about 5 mm.

In some embodiments, the distance between the distal end of the electrode and the surface can be varied by an operator. In some such embodiments, the operator can optionally begin the use of the device with a relatively small distance (e.g., about 1 mm) so that the breakdown potential of the gap is relatively low, and subsequently increase the distance (e.g., to about 5 mm).

### Electrode

The distal end of the electrode is of any suitable electrically conductive material, e.g., aluminum, has any suitable cross-sectional shape that includes the longitudinal axis and any suitable cross-sectional shape that is perpendicular to the longitudinal axis.

In some embodiments, the surface of the distal end of the electrode is conductive. Alternatively, in some embodiments the distal end of the electrode is covered with an insulating material. In some embodiments, the insulating material is a polymer such as PTFE. In some embodiments, the insulating material is a glass coating. In some embodiments, the insulating material is an oxide layer, for example an aluminum electrode having an aluminum oxide layer on the distal end.

In some embodiments, the distal end of the electrode is smooth, for example a smooth flat face or a smooth curved face.

Alternatively, in some embodiments, the distal end of the electrode includes at least one discontinuity. In preferred embodiments, the distal end of the electrode includes not less than four, not less than eight and even not less than twelve discontinuities. Such discontinuities are of any suitable size, typically protruding by not less than about 1 mm from the distal end of the electrode. Preferred discontinuities include pins and similar features: it has been found that, all other things being equal, a distal end of an electrode having multiple discontinuities such as multiple pins (or similar features) provides more uniform plasma generation in proximity of or at a skin surface.

The cross-sectional size and cross-sectional shape of the distal end of the electrode that is perpendicular to the longitudinal axis is any suitable size and shape. In some embodiments, the size is not less than about 0.2 cm² (equivalent to a 0.5 cm diameter circle) and not more than about 80 cm² (equivalent to a 10 cm diameter circle). Preferably the size is not less than about 0.8 cm² (equivalent to a 1 cm diameter circle), and even not less than about 3 cm² (equivalent to a 2 cm diameter circle) and more preferably the size is not less than about 7 cm² (equivalent to a 3 cm diameter circle). Additionally, or alternatively, the size is preferably not more than about 29 cm² (equivalent to a 6 cm diameter circle) and more preferably the size is not more than about 13 cm² (equivalent to a 4 cm diameter circle).

### Potential Relative to Ground

As noted above, an alternating potential is supplied to the electrode. Relative to ground (e.g., earth ground or chassis ground), the magnitude of the potential is any suitable magnitude. In some embodiments, the magnitude of the potential is not less than about 1 kV and even not less than about 2 kV. Additionally, or alternatively, the magnitude of the potential is not more than about 10 kV and even not more than about 5 kV.

### Alternation Rate

As noted above, an alternating potential is supplied to the electrode. The potential alternates at any suitable rate. In some embodiments, the alternation rate is not less than about 5 MHz. In some embodiments, the alternation rate is not less than about 8 MHz, not less than about 9 MHz and even more preferably not less than about 10 MHz. Additionally, or alternatively, in some embodiments the alternation rate is not more than about 600 MHZ, not more than about 433 MHZ and in some embodiments not more than about 300 MHz.

### Power

The power of the current supplied to the electrode is any suitable power, in preferred embodiments not less than about 1 W and not more than about 100 W.

If the power is too low, there will be an insufficient desired effect. Accordingly, in some embodiments the power of the current supplied to the electrode is not less than about 5 W and even not less than about 20 W.

If the power is too high, damage to the surface or to the underlying biological tissue may occur. Accordingly, in some embodiments the power supplied to the electrode is not more than about 80 W and even not more than about 60 W.

The methods according to the teachings herein are implemented using any suitable device or any suitable combination of devices. In preferred embodiments, the method is implemented using a device according to the teachings herein.

### Device According to the Teachings Herein

According to an aspect of some embodiments of the teachings herein, there is provided a device suitable for treating living biological tissue by generating cold plasma in proximity of or at the surface of the biological tissue (in preferred embodiments, generating the cold plasma in accordance with an embodiment of the methods of the teachings herein), the device comprising:
a. an electrode having a longitudinal axis, a proximal end and a distal end, the electrode being part of a circuit;
b. an offset component physically associated with the electrode, the offset component having a distal end, configured so that when the distal end of the offset component contacts a surface of living biological tissue, the distal end of the electrode is maintained at a distance of not less than about 1 mm and more than about 10 mm from the surface, thereby forming a gap between the distal end of the electrode and the surface; and
c. a gas inlet configured for functional association with a gas reservoir,
   wherein the device is configured so that when the distal end of the offset component contacts the surface, gas exiting the gas inlet fills the gap between the distal end of the electrode and the surface.

In preferred embodiments, the electrode is part of an open circuit. The device is configured so that when the distal end of the offset component contacts a surface of living biological tissue and an alternating potential that is supplied to the electrode exceeds the breakdown potential of the gap, discharge occurs between the electrode and the surface, the circuit is closed and cold plasma is generated in the gap in proximity of the surface. As noted above, it is believed that the cold plasma is generated by a glow discharge mechanism.

In some embodiments, the circuit includes a chassis ground.

In some embodiments, the circuit includes an earth ground.

In some embodiments, the electrode, the offset component and the gas inlet are all components of a handpiece of the device. As used herein, a handpiece is a component of the device that can be held in the hand of a healthy average human male, where the human male can hold the handpiece so that the offset component contacts a surface of living biological tissue.

As noted above, when the distal end of the offset component contacts a surface of living biological tissue, the distal end of the electrode is maintained at a distance of not more than about 10 mm from the surface. In some embodiments, the distance is not less than about 1 mm. Additionally or alternatively, in some embodiments the distance is not more than about 8 mm and in some embodiments even not more than about 5 mm.

### Distal End of the Electrode

The distal end of the electrode is of any suitable electrically-conductive material, e.g., aluminum, has any suitable cross-sectional shape that includes the longitudinal axis and any suitable cross-sectional shape that is perpendicular to the longitudinal axis.

In some embodiments, the surface of the distal end of the electrode is conductive. Alternatively, in some embodiments the distal end of the electrode is covered with an insulating material. In some embodiments, the insulating material is a polymer such as PTFE. In some embodiments, the insulating material is a glass coating. In some embodiments, the insulating material is an oxide layer, for example an aluminum electrode having an aluminum oxide layer on the distal end.

In some embodiments, the distal end of the electrode is smooth, for example a smooth flat face or a smooth curved face.

Alternatively, in some embodiments, the distal end of the electrode includes at least one discontinuity. In preferred embodiments, the distal end of the electrode includes not less than four, not less than eight and even not less than twelve discontinuities. Such discontinuities are of any suitable size, typically protruding by not less than 1 mm from the distal end of the electrode. In some embodiments, such discontinuities are not more than about 12 mm. Preferred discontinuities include pins and similar features: it has been found that, all other things being equal, a distal end of an electrode having multiple discontinuities such as multiple pins (or similar features) provides more uniform plasma generation in proximity of or at a skin surface.

The cross-sectional size and cross-sectional shape of the distal end of an electrode that is perpendicular to the longitudinal axis is any suitable size and shape. In some embodiments, the size is not less than about 0.2 cm² (equivalent to a 0.5 cm diameter circle) and not more than about 80 cm² (equivalent to a 10 cm diameter circle). Preferably the size is not less than about 0.8 cm² (equivalent to a 1 cm diameter circle) and even not less than about 3 cm² (equivalent to a 2 cm diameter circle) and more preferably the size is not less than about 7 cm² (equivalent to a 3 cm diameter circle). Additionally or alternatively, the size is preferably not more than about 29 cm² (equivalent to a 6 cm diameter circle) and more preferably the size is not more than about 13 cm² (equivalent to a 4 cm diameter circle).

### Offset Component

A device according to the teachings herein includes an offset component having a distal end, the offset component physically-associated with the electrode. When the distal end of the offset component contacts a surface of living biological tissue, the distal end of the electrode is maintained at a distance of not more than about 10 mm from the surface.

The offset component is preferably of an electrically-insulating material to avoid electrical current flowing from the electrode to the offset component. For example, in some embodiments the offset component is made of a polymer such as PTFE (polytetrafluoroethylene), polyethylene, polypropylene or polycarbonate. In some embodiments, the offset component is made of a transparent material, allowing visual observation of a treated surface without interference or obstruction by the offset component.

In some embodiments, the distal end is configured to allow benign sliding of the distal end along the surface of living biological tissue typically being smooth, for example, to allow benign sliding along the surface of skin or a scalp. As used herein, the term benign sliding means that during the sliding of the distal end along the surface of healthy living biological tissue which the distal end is contacting, the sliding is not painful to a normal healthy person and causes no damage to the surface. For example, in some embodiments the distal end of the offset component is made of PTFE, polyethylene and polypropylene.

In some embodiments, the offset component comprises one or more separate rods or similar components.

In preferred embodiments, the offset component comprises an enclosure having a wall defining a closed proximal portion and an open distal portion comprising a rim thereby defining an open-ended hollow, wherein the distal end of the electrode is located inside the open-ended hollow and is recessed relative to the rim so that when the rim contacts a surface, the distal end of the electrode is at the distance recited above from the surface. In some such embodiments, the gas inlet is directed into the open-ended hollow so that gas exiting the gas inlet enters the open-ended hollow.

In some embodiments, the offset component is an adjustable offset component that is configured to allow varying the distance that is maintained between the surface of living biological tissue and the distal end of the electrode. In some embodiments the offset component is adjustable when the electrode is not energized. Specifically, prior to use and activation of a power supply, a user can adjust the distance that is maintained by the offset component. In some such embodiments, the offset component is not-adjustable when the electrode is energized. Alternatively, in some such embodiments, the offset component is adjustable when the electrode is energized, for example, includes a powered mechanism that allows changing the distance while the electrode is energized.

### Gas Supply

In some embodiments, the device further comprises a gas supply comprising a gas reservoir functionally associated with the gas inlet, the gas supply having at least two states, a first state where no gas flows from the reservoir into the gas inlet and a second state where gas flows from the reservoir through the gas inlet.

The gas reservoir contains any suitable gas or mixture of gases. In some embodiments, the gas in the gas reservoir comprises at least about 50 mole percent, at least about 60 mole percent, at least about 80 mole percent, at least about 90 mole percent and even at least about 95 mole percent of a gas selected from the group consisting of He, Ne, O₂, N₂, Ar and Xe, more preferably a noble gas, most preferably Ar. Additionally or alternatively, in some preferred embodiments the gas in the gas reservoir comprises between about 0.1 and about 10 mole percent N₂ and between about 0.1 and about 10 mole percent O2 and, in a particularly preferred embodiment, comprises at least about 80 mole percent Ar.

### Electrode Cooling

During operation of the device, the temperature of the electrode typically rises.

In some embodiments, the device further comprises cooling components allowing cooling the electrode during operation of the device. In such embodiments, the cooling components are configured to allow maintaining the temperature of the electrode below about 40° C. during operation thereof.

In some embodiments, the device further comprises cooling conduits for transporting a cooling fluid (a gas or a liquid) to the electrode and, in some embodiments, also cooling conduits for transporting a cooling fluid from the electrode.

In some embodiments, the electrode comprises cooling vanes on the surface of the electrode, allowing more efficient cooling of the electrode by a cooling fluid (e.g., a gas) passing along the vanes.

In some embodiments, the electrode comprises cooling-fluid channels that pass through the bulk of the electrode, allowing more efficient cooling of the electrode by a cooling fluid (e.g., a liquid) flowing through the channels.

In some embodiments, the device comprises a cooling fluid driver for driving a cooling fluid to cool the electrode. For example, in some embodiments the cooling fluid driver is a fan or similar component to drive a cooling gas such as air to cool the electrode. Alternatively, in some embodiments the cooling fluid driver is a pump or similar component to drive a cooling liquid such as water to cool the electrode.

In some embodiments, the device comprises cooling conduits for transporting a cooling fluid to the electrode and, in some embodiments, also cooling conduits for transporting a cooling fluid from the electrode and a cooling fluid driver for driving a cooling fluid into the cooling conduit to cool the electrode. For example, in some embodiments the cooling fluid driver is a pump or similar component to drive a cooling liquid such as water to cool the electrode.

In some such embodiments, the device further comprises a cooler to reduce the temperature of a cooling fluid before the cooling fluid is driven to cool the electrode.

In some embodiments, the device is configured so that the gas exiting the gas inlet functions to cool the electrode.

### AC Power Supply Circuit

In some embodiments, the device further comprises an AC power supply circuit functionally-associated with the electrode, the AC power supply circuit configured to supply a potential alternating at a rate of not less than about 5 MHz to the electrode.

The AC power supply circuit is configured to supply any suitable potential relative to ground. In some embodiments, the AC power supply circuit is configured to supply a potential of not less than about 1 kV and even not less than about 2 kV relative to ground to the electrode. Additionally or alternatively, in some embodiments, the AC power supply circuit is configured to supply a potential of not more than about 10 kV and even not more than about 5 kV relative to ground to the electrode.

The AC power supply circuit is configured to supply a potential alternating at a rate of not less than about 5 MHz to the electrode. In some embodiments, the AC power supply circuit is configured to supply a potential alternating at a rate of not less than about 8 MHz, at a rate of not less than about 9 MHz and even at a rate of not less than about 10 MHz to the electrode. Additionally, or alternatively, in some embodiments, the AC power supply circuit is configured to supply a potential alternating at a rate of not more than about 600 MHZ, not more than about 433 MHz and even not more than about 300 MHz.

The AC power supply circuit is configured to supply a potential alternating at a rate of not less than about 5 MHz having any suitable power to the electrode. In some embodiments, the power is not less than about 1 W, preferably not less than about 5 W and even not less than about 20 W. Additionally or alternatively, the power is not is not more than about 100 W, preferably not more than about 80 W and even not more than about 60 W.

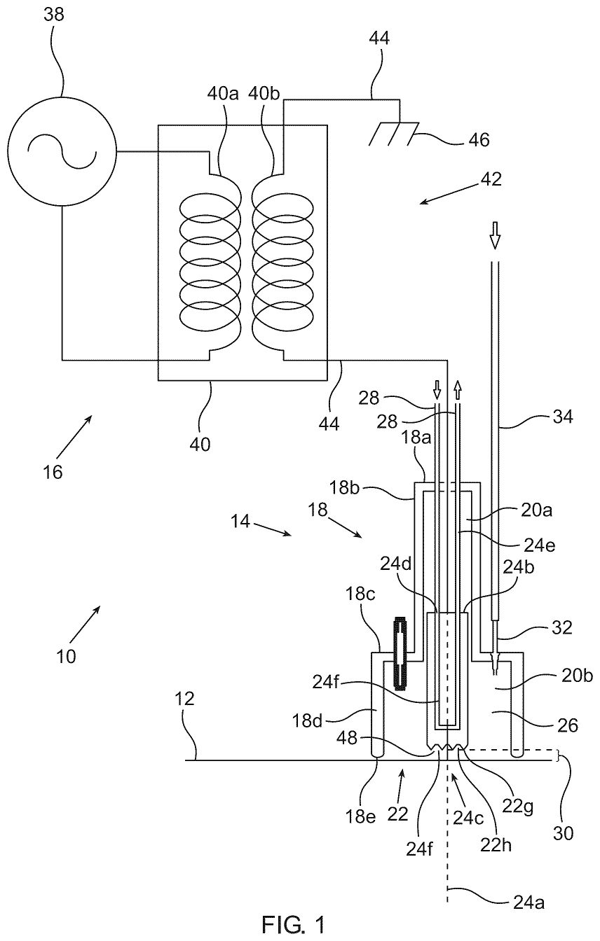

An embodiment of a device according to the teachings herein, device 10, is schematically depicted in FIG. 1 during use for treating a scalp surface 12. Handpiece 14 comprises a hollow body 18 of PTFE with: an end cap 18a, a cylindrical proximal portion 18b, a ring-shaped step portion 18c, and an open-ended cylindrical distal portion 18d. As is apparent from the description herein, body 18 constitutes the offset component of device 10.

Body 18 defines two cylindrical hollows, proximal hollow 20a defined by end cap 18a and proximal portion 18b and open-ended distal hollow 20b defined by step portion 18c and distal portion 18d. Step portion 18c and distal portion 18d can be considered as defining an open-ended enclosure that contains open-ended distal hollow 20b.

The outside of proximal portion 18b of body 18 is configured (including by shape, dimensions and texture) to be easily held by a human hand.

An open distal end 22 of open-ended distal hollow 20b is surrounded by a rim 18e of the distal end of distal portion 18d which is smooth and rounded and thereby configured to allow benign sliding on a biological surface such as skin of a human. Handpiece 14 further comprises inside hollow body 18 a cylindrical electrode 24 of aluminum having a longitudinal axis 24a, a flat proximal end 24b and a not smooth distal end 24c, electrode 24 having a diameter of 3.5 cm and a cross-sectional area of 9.6 cm² perpendicular to axis 24a. Since electrode 24 is cylindrical, the cross-sectional shape of electrode 24 that includes longitudinal axis 24a is a rectangle and the cross-sectional shape of electrode 24 that is perpendicular to longitudinal axis 24a is a circle.

Electrode 24 passes through the hole of ring-shaped step portion 18c so that a proximal portion of electrode 22 is located inside proximal hollow 20a and in intimate contact with the inner walls thereof while a distal portion of electrode 24 is located inside distal hollow 20b and distant from the walls thereof so that the distal portion of electrode 22 inside distal hollow 20b is surrounded by a cylindrical ring-shaped volume 26.

On flat proximal end 24b of electrode 24 are a cooling-fluid inlet 24d and a cooling-fluid outlet 24e in fluid communication one with the other through cooling-fluid channel 24f that passes through the bulk of electrode 22. Functionally-associated with cooling-fluid inlet 22d and cooling-fluid outlet 22e are flexible cooling fluid conduits 28 (e.g., PVC tubing) that are functionally-associated with a liquid cooling pump (not depicted), e.g., from any suitable commercial source such as Tops Industry and Technology Co., Ltd. (International Innovation Park, Zhenhua Road, Yuhua District, Changsha City, Hunan Province, China). The liquid cooling pump is configured as a cooling fluid driver to cyclically pump water as a cooling fluid through cooling fluid conduits 28 and also as a cooler to cool the pumped water.

A distal face 24f of non-flat distal end 24c of electrode 24 includes a 4×4 matrix of sixteen square-based pyramids 24g extending 4 mm from the bulk of electrode 24, each pyramid 24g having a sharp apex 24h, any two neighboring apices 24h separated by 0.7 cm. Electrode 24 is positioned so that distal end 24c recessed relative to rim 18e so that a distance 30 between an imaginary plane defined by rim 18e of the distal end of distal portion 18d of body 18 and apices 24h is 5 mm. As a result, when rim 18e contacts a skin surface such as scalp surface 12, the distance between the surface and apices 22h is 5 mm.

Handpiece 14 further comprises a gas inlet 32 that passes through ring-shaped step portion 18c of body 18. Gas inlet 32 is functionally-associated with a gas-supply tube 34 which is functionally-associated with a gas reservoir (not depicted), such as a gas cylinder. When a valve of the gas reservoir is opened, gas flows from the gas reservoir, through gas-supply tube 34 and through gas inlet 32 into cylindrical ring-shaped volume 26 that surrounds the distal portion of electrode 24 inside distal hollow 20b.

Handpiece 14 further comprises a non-contact thermometer 36 that passes through ring-shaped step portion 18c of body 18. Non-contact thermometer 36 is directed so as to measure the temperature of a surface that is surrounded by a rim 18e of the distal end of distal portion 18d of body 18 and apparent through open distal end 22 of open-ended distal hollow 20b.

AC power supply circuit 16 of device 10 comprises an AC power supply 38 functionally-associated with a transformer 40. AC power supply circuit 16 further comprises an open circuit 42 comprising a secondary winding 40b of transformer 40 and a ground 44 (in device 10, a chassis ground) functionally-associated with electrode 24 through lead 46.

For use of device 10, the liquid cooling pump is activated so that a cooling liquid such as water maintained at 20° C. continuously passes from the liquid cooling pump into cooling fluid conduits 28, in through cooling-fluid inlet 24d into and through cooling-fluid channel 24f, out through cooling-fluid outlet 24e and back through cooling fluid conduit 28 to liquid cooling pump, thereby maintaining electrode 24 at close to 20° C. A valve of the gas source is opened so that gas (e.g., argon) passes through gas-supply tube 34 and through gas nozzle 32 into cylindrical ring-shaped volume 26 that surrounds the distal portion of electrode 24 inside distal hollow 20b.

A user holds handpiece 14 with one hand so that rim 18e presses against scalp surface 12. As a result, distal end 24c of electrode 24 is positioned 5 mm from a surface such as scalp surface 12, forming a gap 48 between electrode 24 and surface 12. Gas from gas nozzle 32 that fills cylindrical ring-shaped volume 26 and gap 48 constitutes a gaseous atmosphere.

The user activates AC power supply 38 through a non-depicted controller. Under control of the controller, AC power supply 38 provides an alternating potential having a user-selected frequency, power and potential to primary winding 40a of transformer 40. When the potential supplied to the electrode exceeds the breakdown potential of gap 48, discharge occurs between the electrode and the surface, circuit 42 is closed and cold plasma is generated in gap 48 and thereby in proximity of and at surface 12. Preferably cold plasma generation is accompanied by generation of plasma streamers between apices 24h of electrode 24 and surface 12.

### EXPERIMENTAL

An embodiment of a device according to the teachings herein as depicted in FIG. 1 is made where the offset component is set to maintain the distal end of the electrode at a distance of 5 mm from a scalp.

Ten human male subjects aged 20 to 27 all exhibiting early stages male-pattern hair loss with a distinct hair loss at least on the crown of the head are treated.

Before treatment, the diameters of ten hairs randomly harvested from each one of the ten subjects is determined. The average hair diameter for each one of the ten subjects is found to be between 40 and 60 micrometers.

99.9% argon gas is allowed to continuously flow into the enclosure through the gas inlet and the electrode is maintained at 20° C. by the continuous flow of 20° C. water through the cooling fluid channels. The AC power supply circuit is set to supply a 4 kV potential alternating at 20 MHz with a current power of 30 W to the electrode. Each subject is treated with two sessions a week, each session being 15 minutes long during which time the entire area of hair loss is treated. During treatment, numerous plasma streamers are observed between the distal end of the electrode and the scalp, the plasma streamers being in constant motion.

It is believed that despite 99.9% argon being supplied into the enclosure, the sliding of the handpiece along the scalp surface allows air (˜80% nitrogen and ˜20% oxygen) to leak into the enclosure and into the gap between the distal end of the electrode and the scalp surface.

After 1 month (8 treatment sessions) it is observed that the area of hair loss of all of the subjects is smaller and less apparent. The average hair diameter of all subjects is found to have increased to be between 60 and 80 micrometers.

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof. As used herein, the indefinite articles “a” and “an” mean “at least one” or “one or more” unless the context clearly dictates otherwise.

As used herein, when a numerical value is preceded by the term “about”, the term “about” is intended to indicate +/−10%. As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention. Section headings are used herein to ease understanding of the specification and should not be construed as necessarily limiting.

1. A method of treating living biological tissue of a subject, comprising:
providing an electrode having a longitudinal axis and a distal end;
in the presence of a gaseous atmosphere, placing said distal end of said electrode in proximity of a surface of living tissue with said longitudinal axis directed towards said surface so that said distal end of said electrode is at a distance of not less than about 1 mm and not more than about 10 mm from said surface so as to form a gap between said distal end of said electrode and said surface wherein said surface thereby functions as a ground electrode; and
supplying to said electrode a potential alternating at a rate of not less than about 5 MHz with a power of not less than about 1 W and not more than about 100 W,
wherein said potential supplied to said electrode exceeds a breakdown potential of said gap thereby generating cold plasma in said gap in proximity of said surface which generated cold plasma treats said biological tissue.

2. The method of claim 1, wherein said generating cold plasma in proximity of said surface is accompanied by formation of plasma streamers between said distal end of said electrode and said surface.

3. The method of claim 1, wherein said gaseous atmosphere is at least about 50 mole percent Ar.

4. The method of claim 1, wherein said gaseous atmosphere is between about 0.1 and about 10 mole percent N₂ and between about 0.1 and about 10 mole percent O₂.

5. The method of claim 1, wherein said distance is not more than about 8 mm.

6. The method of claim 1, wherein said distance is not more than about 5 mm.

7. The method of claim 1, wherein a cross-sectional size of said distal end of said electrode that is perpendicular to said longitudinal axis is not less than about 0.8 cm² and not more than about 80 cm².

8. The method of claim 1, wherein a magnitude of said potential is not less than about 1 kV and not more than about 10 kV.

9. The method of claim 1, wherein said rate at which said potential alternates is not less than about 8 MHz.

10. The method of claim 1, wherein said power is not less than about 5 W and not more than about 80 W.

11. A device suitable for treating living biological tissue by generating cold plasma in proximity of the surface of the biological tissue, the device comprising:
a. an electrode having a longitudinal axis, a proximal end and a distal end, said electrode being part of a circuit;
b. an offset component physically associated with said electrode, said offset component having a distal end, configured so that when said distal end of said offset component contacts a surface of living biological tissue, said distal end of said electrode is maintained at a distance of not less than about 1 mm and more than about 10 mm from the surface, thereby forming a gap between said distal end of said electrode and the surface; and
c. a gas inlet configured for functional association with a gas reservoir,
wherein the device is configured so that when said distal end of said offset component contacts a surface, gas exiting said gas inlet fills said gap between said distal end of said electrode and the surface.

12. The device of claim 11, wherein said electrode, said offset component and said gas inlet are all components of a handpiece of the device.

13. The device of claim 11, wherein the distance is not more than about 8 mm.

14. The device of claim 11, wherein said distal end of said electrode is not smooth and comprises at least one discontinuity.

15. The device of claim 14, wherein said distal end of said electrode comprises not less than four discontinuities.

16. The device of claim 11, where a cross-sectional size of said distal end of said electrode that is perpendicular to said longitudinal axis is not less than about 0.8 cm² and not more than about 80 cm².

17. The device of claim 11, wherein a distal end of said offset component is configured to allow benign sliding along the surface of living biological tissue.

18. The device of claim 11, wherein said offset component comprises an enclosure having a wall defining a closed proximal portion and an open distal portion comprising a rim, thereby defining an open-ended hollow, wherein said distal end of said electrode is located inside said open-ended hollow and is recessed relative to said rim so that when said rim contacts a surface, said distal end of said electrode is at said distance from the surface.

19. The device of claim 18, wherein said gas inlet is directed into said open-ended hollow so that gas exiting said gas inlet enters said open-ended hollow.

20. The device of claim 11, further comprising an AC power supply circuit functionally associated with said electrode, said AC power supply circuit configured to supply a potential alternating at a rate of not less than about 5 MHz to said electrode.
